(19) United States
(12) Patent Application Publication
(10) Pub. No.: US 2010/0099170 A1
(43) Pub. Date: Apr. 22, 2010

(54) METHODS OF CONTROLLING OPEN ALGAL BIOREACTORS

(76) Inventor: Deepak Aswani, Westland, MI (US)

Correspondence Address: BROWN & MICHAELS, PC;400 M & T BANK BUILDING, 118 NORTH TIOGA ST, ITHACA, NY, 14850, US

(21) Appl. No.: 12/254,226

(22) Filed: Oct. 20, 2008

(51) Int. Cl. C12N 1/12

(52) U.S. Cl. 435/257.1

(57) ABSTRACT

A method controls growth in an open algae cultivation system. The cultivation system includes a high-yield species and at least one invasive native species. The method includes adjusting at least one parameter of the system to a first value such that a high-growth condition for the high-yield species is produced in the open algae cultivation system. The method also includes adjusting the parameter to a second value different from the first value such that a high-dominance condition for the high-yield species is produced in the open algae cultivation system. In one embodiment, the method includes adjusting from the first value to the second value when the concentration of the high-yield species reaches a lower limit and adjusting from the second value to the first value when the concentration of the high-yield species reaches an upper limit.

### BACKGROUND OF THE INVENTION

1. Field of the Invention

The invention pertains to the field of control systems. More particularly, the invention pertains to control systems and methods for open cultivation of algae.

2. Description of Related Art

The control of inputs to an algae bioreactor system is critical in influencing the growth and properties of the algae. Most demonstrations of both closed and open algae cultivation use some form of bioreactor control. Control systems include a device or set of devices that manage, command, direct, or regulate the behavior of a system of interest. Control systems are commonly implemented using sensors and actuators in conjunction with a computer, often including an embedded system, able to make periodic sensor measurements which are used for calculations. These calculations are used for commanding the actuators that directly influence the behavior of the system of interest. Since computers are central to the function of control systems, the effectiveness of a control system is determined by the algorithms and software used.

Most control systems for algae cultivation are based on Programmable Logic Controllers (PLC) and leverage control systems technology used for fermentation bioreactors. AlgaeLink (Roosendaal, NL) markets a PLC-based control system for closed bioreactors for algae cultivation. Although modified control systems derived from fermentation applications do facilitate algae growth via control of environmental conditions, they do not address one of the unique challenges for open algae cultivation, the presence of invasive competitors or predators in the bioreactor. Open algae cultivation is usually more desirable than closed algae cultivation for cost reasons. However, invasive competitors or predators are a drawback for conventional control systems applied to open algae cultivation. Conventional control systems focus on regulating environmental parameters to steady state values to promote algae growth. In U.S. Pat. No. 4,438,591, issued to Kessler, dissolved salts or nutrients, temperature, pH, liquid turbulence, and light intensity or light spectrum are controlled to enhance the growth of algae that is to be harvested. In U.S. Pat. No. 5,541,056, issued to Huntley et al., nutrient delivery, liquid medium inflow or outflow, and fluid turbulence are controlled to facilitate algae growth. In U.S. Pat. No. 7,156,985, issued to Frisch, the regulation of temperature to favor algae growth is advocated.

Potential real-time control choices that may be made by actuators include, but are not limited to, inflow of brackish water, inflow of fresh water, inflow of CO₂ aeration, paddle speed or mix rate, agro-human waste inflow, harvest rate outflow, chemical additive inflow, and UV radiation. Salinity, temperature, pH, and CO₂ concentration all affect algal growth rate. The paddle speed or mix rate influences nutrient distribution and photo-modulation. Addition of agro-human waste increases nitrate and phosphate concentrations. The harvest rate impacts the pond depth and algal concentration. Concentrations of chemicals additives, including limiting nutrients such as silicates or phosphates, depending on algae type, also affect algal growth rate. UV radiation has an adverse effect on algal growth.

Potential uncontrollable external inputs or disturbances in an open algal bioreactor include, but are not limited to, sunlight, precipitation, humidity, ambient temperature, and entry of invasive competitors or predators.

Potential real-time measurements that may be made by sensors include, but are not limited to, pond temperature (preferably measured by a thermocouple), salinity (preferably measured by conductance), pH (preferably by a glass electrode), water level (preferably measured by a flotation sensor), sunlight intensity (preferably measured by a photovoltaic cell), oxygen concentration (measured by an O₂ sensor), carbon dioxide concentration (measured by a CO₂ sensor), algae concentration (preferably calculated from a mass rate measurement of harvested dry mass), and lipid concentration (preferably calculated from a flow rate measurement of extracted oil).

A high-level control objective for algae cultivation is to maximize the yield of a particular species of algae. For biodiesel production, this species may have a high content of lipids. For nutritional cultivation, this species may have a high content of certain vitamins or amino acids. There may be other specialty species of interest depending on needs. Conventional methods identify how controlling environmental conditions can increase the rate of algae growth. However, there is a need in the art for a control system that addresses the presence of invasive competitors or predators relative to a species of interest or high-yield species. Dealing with invasive competitors or predators is a major challenge in achieving satisfactory yield in open pond cultivation of algae.

### SUMMARY OF THE INVENTION

A method controls growth in an open algae cultivation system in a first embodiment of the present invention. The cultivation system includes a high-yield species and at least one invasive native species. The method includes adjusting at least one parameter of the system to a first value such that a high-growth condition for the high-yield species is produced in the open algae cultivation system. The method also includes adjusting the parameter to a second value different from the first value such that a high-dominance condition for the high-yield species is produced in the open algae cultivation system. In one embodiment, the method includes adjusting from the first value to the second value when the concentration of the high-yield species reaches a lower limit and adjusting from the second value to the first value when the concentration of the high-yield species reaches an upper limit.

A method controls growth in an open algae cultivation system in a second embodiment of the present invention. The cultivation system includes a high-yield species and at least one invasive native species. The method includes maintaining the system at a high-growth condition for the high-yield species for a first portion of time. The method also includes maintaining the system at a high-dominance condition for the high-yield species for a second portion of time.

### BRIEF DESCRIPTION OF THE DRAWINGS

FIG. 1 shows an overview of algal biodiesel creation from pond to pump.

FIG. 2 shows a conventional “high-rate pond” or open bioreactor formed by a racetrack trench.

FIG. 3 shows a hypothetical species dominance depicted with respect to the space of system states.

FIG. 4 shows a hypothetical species growth rate depicted with respect to the space of system states.

FIG. 5 shows a high-level switching control scheme for open algae cultivation, with direct mode selection, implemented as sequential decisions in an embodiment of the present invention.

FIG. 6 shows a high-level switching control scheme for open algae cultivation, with indirect mode selection via duty cycle, implemented as sequential decisions in an embodiment of the present invention.

FIG. 7 shows a high-level switching control scheme for open algae cultivation, with indirect mode selection via duty cycle, implemented as a proportional-integral controller in an embodiment of the present invention.

FIG. 8 shows a high-level and low-level control scheme for open algae cultivation with potential control choices in an embodiment of the present invention.

FIG. 9 shows implementation of the embodiment of FIG. 8 in an open pond configuration, with a single control choice of fertilizer concentration.

FIG. 10 shows a prior art control scheme for open algae cultivation in an open pond configuration, with a single control choice of fertilizer concentration.

FIG. 11 shows a simulation of the prior art scheme of FIG. 10, operated at the high-growth mode.

FIG. 12 shows a simulation of the prior art scheme of FIG. 10, operated at the high-dominance mode.

FIG. 13 shows a simulation of the method of FIG. 5 and FIG. 6, with mode switching between high-growth and high-dominance.

FIG. 14 shows a simulation of the method of FIG. 7, with mode switching between high-growth and high-dominance.

### DETAILED DESCRIPTION OF THE INVENTION

Algae productivity is not necessarily correlated with species dominance or persistence. Control of resources and environmental conditions may be used to facilitate the dominance of high-yield species in open cultivation, which may not necessarily coincide with high growth rates.

Methods of the present invention address both the growth rate of algae and the dominance of algae through control of resources and environmental conditions such that the methods are particularly applicable when biologists have successfully isolated, bred, or engineered high-yield strains that can stably coexist with the invasive native species in open bioreactors in a target cultivation environment. For example, one high-yield strain recommended for biodiesel cultivation is the green algae Monoraphidium minutum. Monoraphidium has been cited as being sensitive to invasion by more dominant species of algae. Sample collection from the Gulf of Bothnia of the Baltic Sea showed that Monoraphidium was the third-most abundant species at approximately 18% biomass composition, lower than both Synechococcus and M. rubrum. This indicates that Monoraphidium may be a suitable candidate for cultivation around the Baltic Sea where it is known to stably coexist. The green algae Monoraphidium may not be suitable for environments where it may be challenged for stable coexistence, such as the Sonoran Desert, which is typically abundant with cyanobacteria algae. Another example of a recommended high-yield strain is the freshwater diatom Cyclotella. Cyclotella is an algae that naturally grows in the waters of Lake Michigan alongside competitors Fragilaria, Asterionella, Synedra, and Tabellaria. Under the right environmental and resource conditions, the high-yield strains dominate among the invasive native species. Experimental cultivation with other species using conventional control methods has shown that Cyclotella was found to be lost or greatly diminished.

Cyclotella is found to be regionally dominant to Asterionella as a result of natural resource gradients. Therefore, control of environmental and resource conditions may be used to provide protection from the elimination of the high-yield strains. However, this could be a costly solution in terms of net yield, and possibly control cost or effort. The region of operation where the high-yield strain is dominant may not necessarily coincide with the region of high-growth rates. This could thus compromise yield. A region of high-growth rate of the high-yield species may coincide with a region of higher growth rate for an invasive native species. The regions where the high-yield species shows dominance may be at low growth rates. Therefore, rather than exclusively establishing dominance of the high-yield species, it is of interest to maximize net lipid yield without elimination of the high-yield species.

A method of the present invention includes switching cultivation conditions between conditions of high-growth rate of the high-yield species and conditions of high-dominance of the high-yield species. Under the high-growth rate conditions, the high-yield species quickly grows, which produces a large quantity of the substances of interest such as lipids or vitamins, depending on the objective of cultivation. The high-dominance conditions ensure that the presence of the high-yield species is not diminished to a point that production of the substances of interest is compromised.

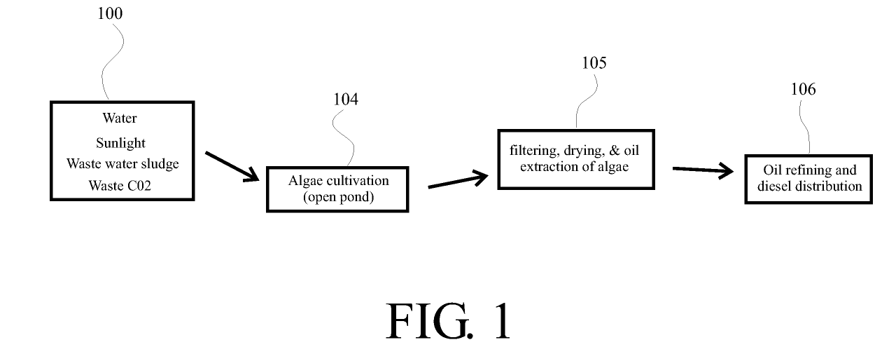

FIG. 1 depicts a process of targeted algae growth. Certain species of algae are harvested for their high lipid content to produce biodiesel. Inputs 100 such as water 107, sunlight 101, waste water sludge/nutrients 102, and waste carbon dioxide 103 are fed to the open pond 104. This mixture is harvested and then is filtered and dried and its oil is extracted 105. Finally the extracted crude oil is processed and refined to produce diesel fuel 106.

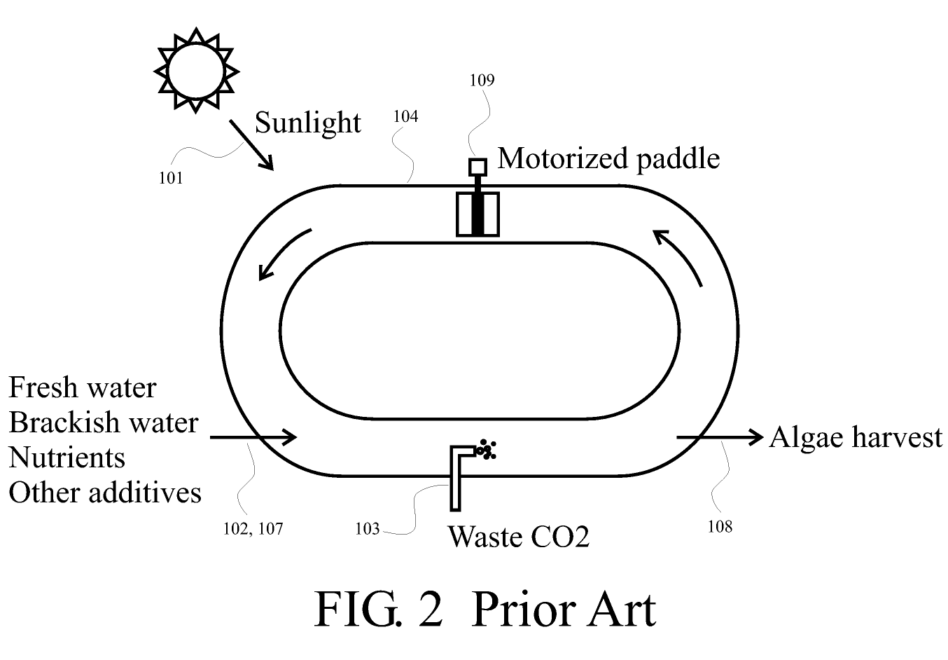

FIG. 2 shows more detail of a preferred pond cultivation environment for algae. Often a pond mixing device such as a motorized paddle 109 is used to maintain a consistent mixture with minimal resource gradients. The pond 104 has an entry point for dilution inflow 107 of fresh water, brackish water, nutrients, or other additives 102 and an exit point for harvest outflow 108.

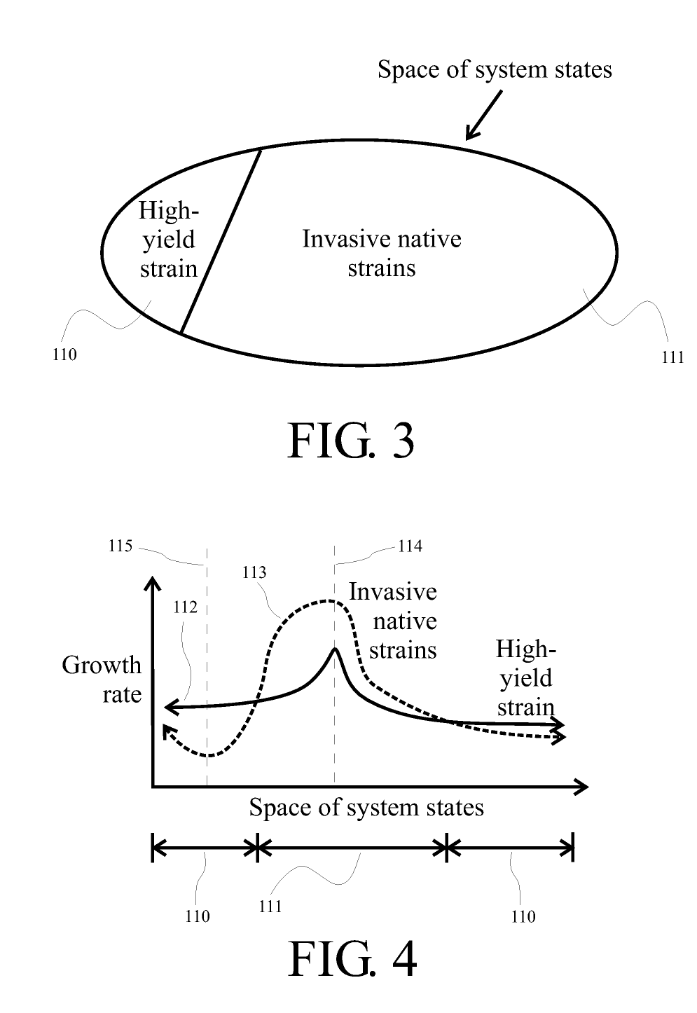

FIG. 3 shows that the high-yield species are often out-competed by invasive or predatory species under certain spaces of states 111. The term “spaces of states” as used herein refers to the continuum of one or a combination of conditions of the algae which affect the growth rate of algae in the bioreactor. The species of interest, with high lipid content in some embodiments, is referred to as high-yield species. However, there are typically certain spaces of states 110 where the high-yield species maintains dominance.

FIG. 4 shows that dominance of the high-yield species is described by the space of states where the growth rate 112 of the high-yield species exceeds the growth rate 113 of the invasive species. Dominance of the invasive species is described by the space of states 111 where the growth rate 113 of the invasive species exceeds the growth rate 112 of the high-yield species, as shown in FIG. 4. The peak growth rate of the high-yield species is shown at 114. The peak dominance of the high-yield species is shown at 115. As depicted in FIG. 4, it is possible that the peak growth rate and peak dominance of the high-yield species do not coincide.

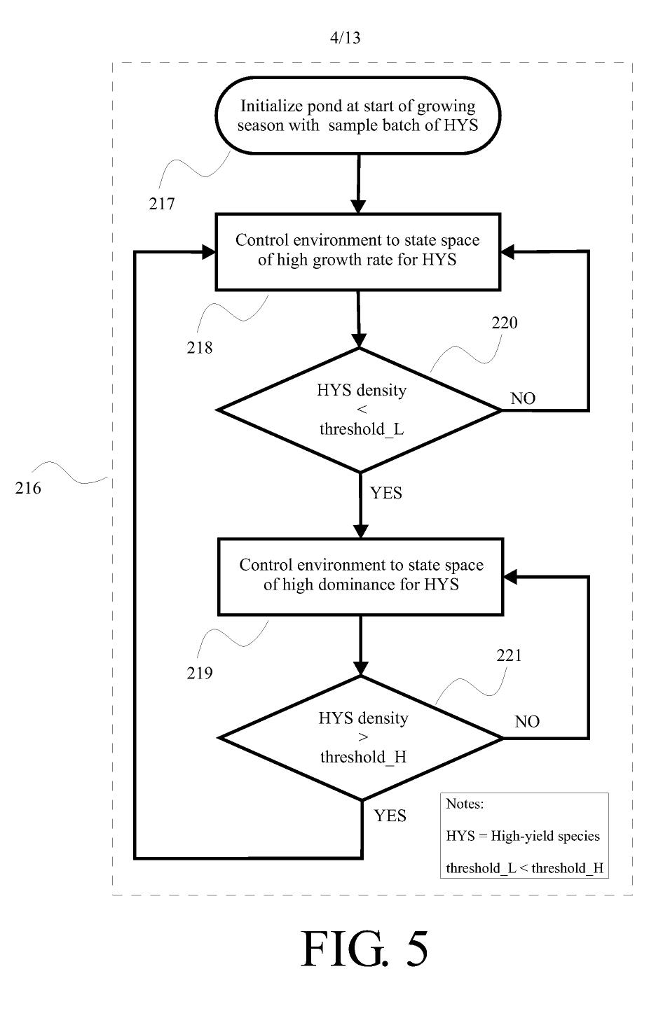

FIG. 5 shows a first embodiment of a method of the present convention 216 of controlling the open algae cultivation, where the mode of operation is directly determined based on feedback of the measured density of the high-yield species. The pond is initialized 217 at the start of the growing season with a sample batch of the high-yield species. The pond is controlled 218 to a state space of high-growth rate for the high-yield species. Although a high-growth condition may be any condition which promotes an above-average or high rate of growth of the high-yield species within the spirit of the present invention, a preferred high-growth condition is the condition for the highest growth rate for the high-yield species (114 in FIG. 4) for a given control variable or set of control variables. An inquiry 220 is made regarding the density of the high-yield species. If the measured density of the high-yield species is not below a calibratable threshold, threshold_L, then a decision is made to continue the high-growth rate operation 218. If the measured density of the high-yield species is below threshold_L, then a decision is made to transition to a high-dominance mode 219 for the high-yield species. Although a high-dominance condition may be any condition which promotes a faster growth of the high-yield species than of an invasive native species within the spirit of the present invention, a preferred high-dominance condition is the condition where the ratio of high-yield species growth rate to invasive native species growth rate is maximized (115 of FIG. 4) for a given control variable or set of control variables. When more than one invasive native species is present, a preferred high-dominance condition is the condition where the ratio of the high-yield species growth rate to the sum of the invasive native species growth rates is maximized. Another inquiry 221 is made regarding the density of the high-yield species. If the measured density of the high-yield species is not above another calibratable threshold, threshold_H, where threshold_H is greater than threshold_L, then a decision is made to continue the high-dominance mode of operation 219. If the measured density of the high-yield species is above threshold_H, then a decision is made to transition to the high-growth rate mode operation 218.

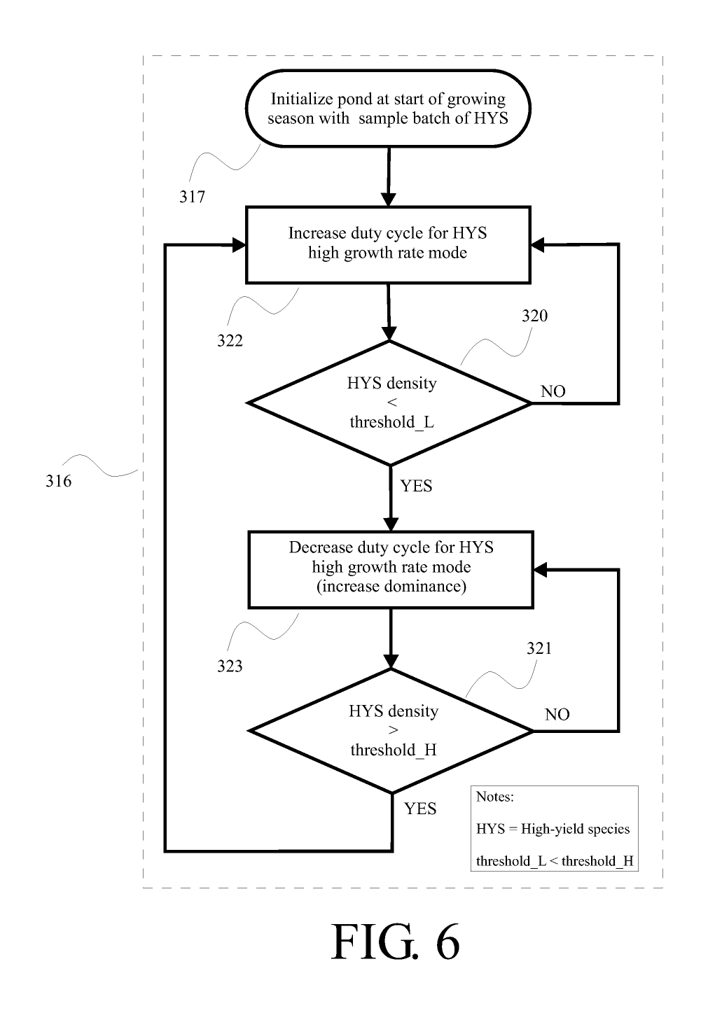

FIG. 6 shows a second embodiment of a method of the present invention 316 of controlling the open algae cultivation, where the mode of operation is determined based on a periodic square wave, for which the duty cycle is increased or decreased based on feedback of the measured density of the high-yield species. This embodiment is similar to the embodiment of FIG. 5, except that a periodic square wave determines whether the high-growth mode (preferably at 114 of FIG. 4) or the high-dominance mode (preferably at 115 of FIG. 4) is followed. A low level of the square wave designates a high-dominance mode and a high level of the square wave designates a high-growth mode. On this basis, the time in residence at each mode per period of the square wave is designated by the duty cycle, defined as the ratio of time in residence at a high level divided by the period. First the pond is initialized 317 at the start of the growing season with the high-yield species. The fixed period and the initial condition for the duty cycle are preferably selected such that the lipid yield rate is optimized while maintaining dominance of the high-yield species. The core alternating mode behavior provided in the embodiment of FIG. 5 is inherent to the fixed period and initial condition of the duty cycle in the embodiment of FIG. 6. Unlike in the embodiment of FIG. 5, the decision logic provides an adaptive function to adjust the duty cycle if the duty cycle assumptions do not hold. One example of the duty cycle assumptions not holding would be a gradual reduction in high-yield species dominance. The duty cycle is increased 322 for a high-growth rate for the high-yield species. The periodicity for the duty cycle is preferably chosen such that the duty cycle may be adjusted frequently enough to maintain adequate control but not frequently enough that the system spends a significant fraction of time in undesirable conditions between the high-growth and high-dominance modes. An inquiry 320 is made regarding the density of the high-yield species. If the measured density of the high-yield species is not below a calibratable threshold, threshold_L, then a decision is made to continue increasing 322 the high-growth rate duty cycle. If the measured density of the high-yield species is below threshold_L, then a decision is made to decrease 323 the high-growth rate duty cycle. Another inquiry 321 is made regarding the density of the high-yield species. If the measured density of the high-yield species is not above another calibratable threshold, threshold_H, where threshold_H is greater than threshold_L, then a decision is made to continue decreasing 323 the high-growth rate duty cycle. If the measured density of the high-yield species is above threshold_H, then a decision is made to increase 322 the high-growth rate duty cycle.

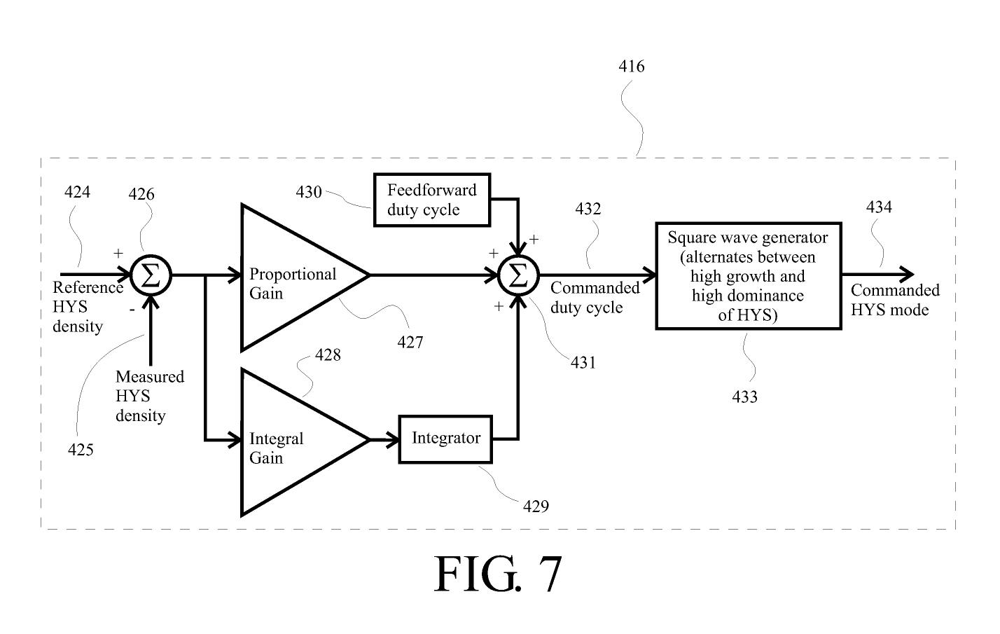

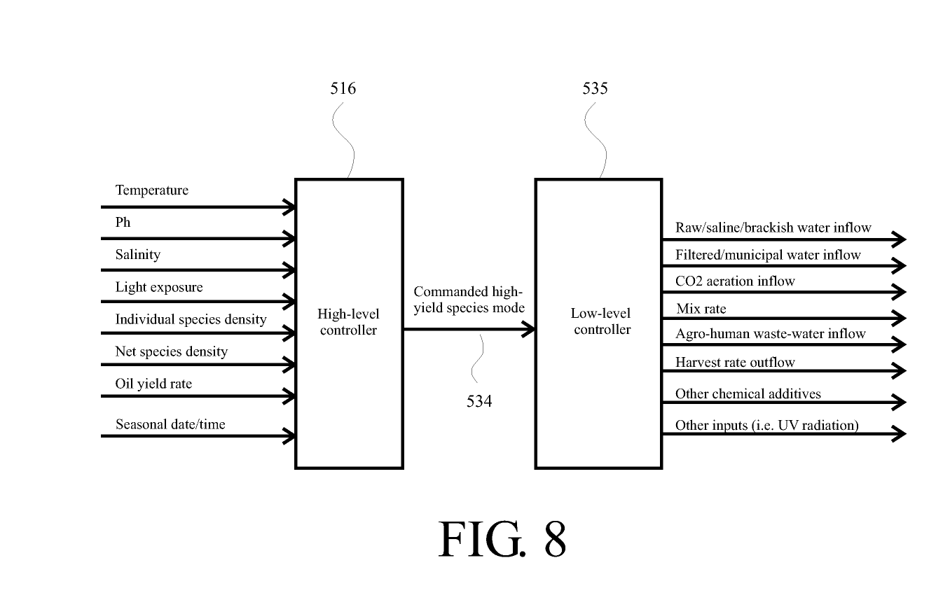

FIG. 7 shows implementation of proportional-integral (PI) control of the sequential decision implementation of FIG. 6 in a third embodiment of the present invention 416. A difference between the reference high-yield species density 424 and the measured high-yield species density 425 is taken in a first summation 426. This quantity is multiplied by an integral gain 428 and integrated using an integrator 429. The difference from summation 426 is also multiplied by a proportional gain 427 and added to both the integrated quantity from 429 and a feedforward duty cycle 430 in a second summation 431. This results in a commanded duty cycle 432, which is used to select the modes 434, either a high dominance condition or a high growth condition, via a square wave generator 433. The periodicity for the duty cycle is preferably chosen such that the duty cycle may be adjusted frequently enough to maintain adequate control but not frequently enough that the system spends a significant fraction of time in undesirable conditions between the high-growth and high-dominance modes. Additionally, the PI controller is preferably calibrated for very slow closed loop dynamics in order to avoid potential drastic changes in the duty cycle.

FIG. 8 shows a generalized implementation of a controller of the present invention. The high-level mode selection decision 516, which may be, but is not limited to, one of the previously described methods 216, 316, 416, is determined by the density of the individual species. However, other information about current inputs or states may be used to supplement the decision when operating outside of the typical regions of operation. The high-level mode decision 516 of whether to operate in high-growth or high-dominance may be executed through the use of multiple control choices. This execution takes place in a low-level controller 535. The high-growth and high-dominance modes are commanded 534 from the high-level controller 516 to the low-level controller 535. The low-level controller contains a mapping of the state space to the high-growth mode and the high-dominance mode. The control choices may include, but are not limited to, raw water inflow, filtered water inflow, CO₂ aeration inflow, mix rate, agro-human waste-water inflow, harvest rate outflow, inflow of other chemical additives such as fertilizer, and other inputs such as UV radiation. The control choices are used in an attempt to reach the mapped state space. Adjustment by some low-level tracking controllers may be necessary to accomplish this if the controlled state space is not a subset of system inputs.

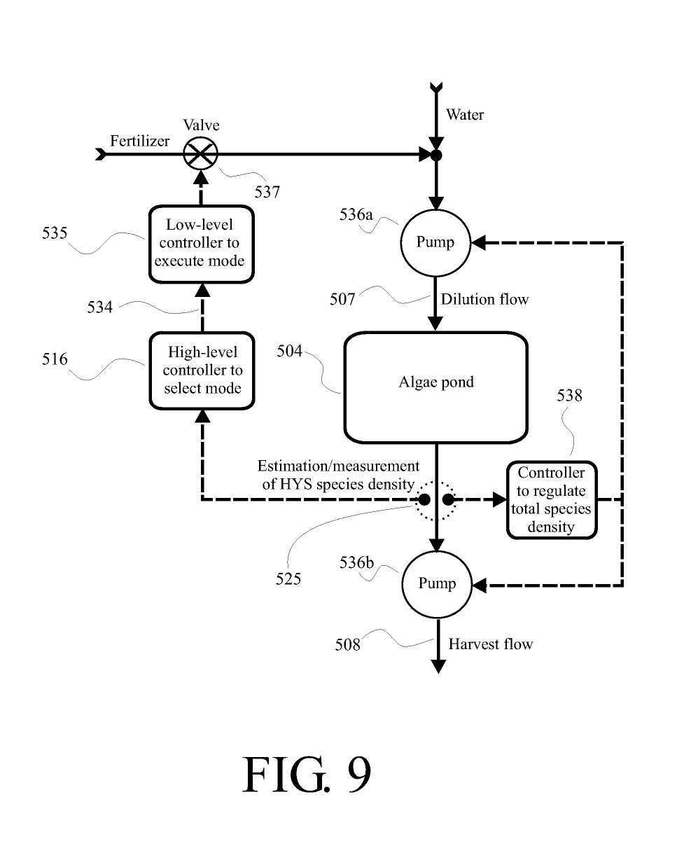

FIG. 9 shows an example of a method of the present invention to control open algae cultivation integrated into an open bioreactor control system. The high-level controller 516 is in the form of the controllers 216, 316, or 416 of FIG. 5, FIG. 6, or FIG. 7, respectively. The high-level controller uses feedback of the measured density 525 of the high-yield species. In FIG. 9, only one control choice, fertilizer concentration, is applied using a valve 537, although multiple control choices with multiple valves may be used in a single controller within the spirit of the present invention. Therefore, the high-growth and high-dominance modes are realized only through two levels of fertilizer concentration. This means that the low-level controller is a mapping of high-growth to one fertilizer concentration and high-dominance to another fertilizer concentration. The delivered fertilizer is mixed with the inflowing water and pumped 536a as a dilution flow 507 into the open pond 504. Simultaneously, the harvest outflow 508 is pumped 536b out of the open pond. The pumps 536a, 536b are preferably operated by a controller 538 that regulates the total species density so that the pond density is appropriate to facilitate growth.

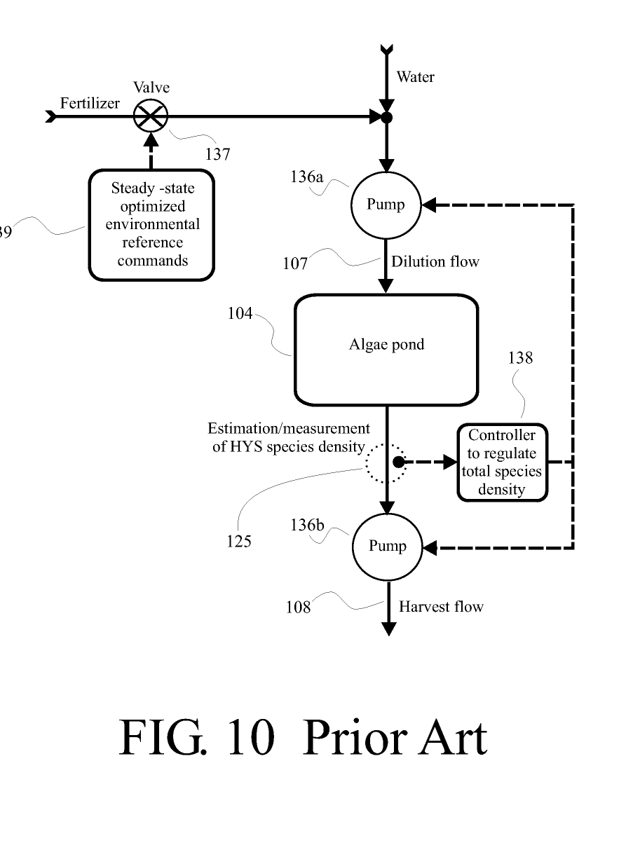

FIG. 10 shows a prior art method to control open algae cultivation 139, in a similar environment to that of FIG. 9. Fertilizer concentration is applied using a valve 137. The delivered fertilizer is mixed with the inflowing water and pumped 136a as a dilution flow 107 into the open pond 104. Simultaneously, the harvest outflow 108 is pumped 136b out of the open pond. An important difference is that the control choice of fertilizer is a feedforward command in this control system, which is determined only by environmental inputs, without any state feedback such as a measured density of the high-yield species 125. As a result, the prior art controller 138 may only control the system for algae growth in either a high-growth mode or a high-dominance mode if those modes are not coincident.

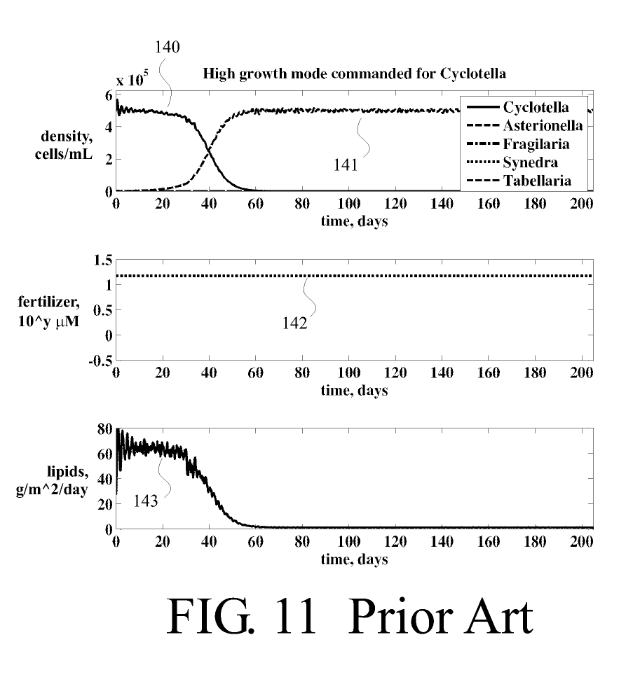

FIG. 11 shows simulation results of the prior art method of FIG. 10 as applied to five fresh water algae species found in Lake Michigan: Cyclotella, Asterionella, Fragilaria, Synedra, and Tabellaria. Cyclotella is the high-yield species in terms of lipids for diesel production. Silicate fertilizer 142 is applied at a high concentration to result in a high-growth mode for Cyclotella. After about 40 days of growth, the density 140 of Cyclotella diminishes and an invasive native strain, Asterionella (density 141), overtakes the open culture. The densities of Fragilaria, Synedra, and Tabellaria remain at minimal levels throughout the simulation such that they are not visible in FIG. 11. The lipid yield 143 is initially between 60 and 80 g/m² per day. As the high-yield species diminishes, the lipid yield 143 of the pond is greatly reduced until essentially no lipid is being produced.

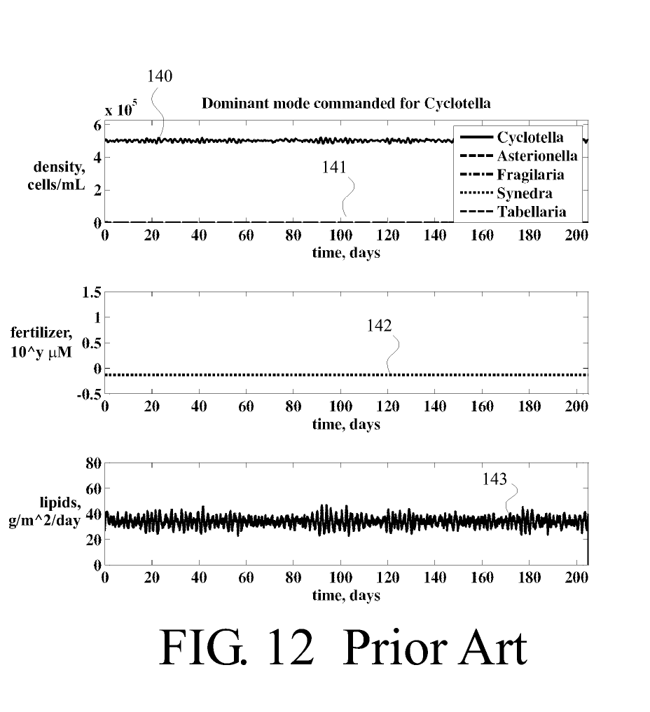

FIG. 12 shows simulation results of the prior art method of FIG. 10 as applied to the same five fresh water algae species in a high-dominance mode. Silicate fertilizer is applied at a low concentration 142 to result in the high-dominance mode for Cyclotella. Although an invasive native strain, Asterionella, never overtakes the high-yield species Cyclotella, the lipid yield 143 is consistently moderate around 35 g/m² per day in comparison to the short-lived yield of about 70 g/m² per day seen with the high growth mode in FIG. 11. The densities of Fragilaria, Synedra, and Tabellaria remain at minimal levels throughout the simulation such that they are also not visible in FIG. 12. Therefore, there is a tradeoff between dominance of the high-yield species Cyclotella and growth rate of Cyclotella.

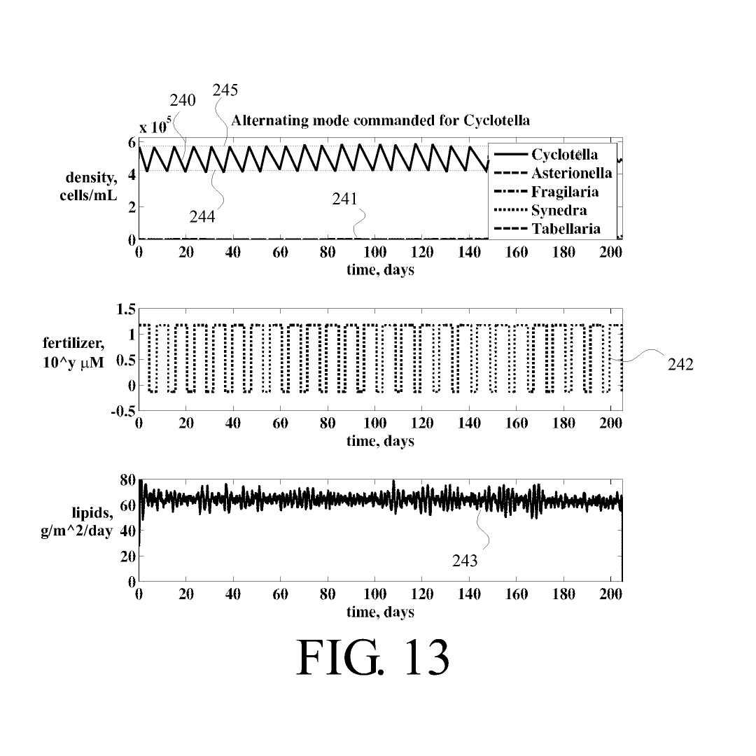

FIG. 13 shows simulation results using control methods of the first or second embodiment of the present invention as applied to the same five fresh water algae species. Silicate fertilizer is initially applied at a high concentration 242 to result in a high-growth mode for Cyclotella. Eventually the high-yield species concentration 240 of Cyclotella declines. When the high-yield species concentration 240 of Cyclotella falls to a low threshold 244, the SiO₂ fertilizer is applied at a low concentration 242 to result in a high-dominance mode for Cyclotella. Eventually the high-yield species concentration 240 of Cyclotella is recovered. The high-dominance mode continues until the high-yield species concentration 240 of Cyclotella reaches a high threshold 245. At this point, the high-growth mode is again applied. This process continues indefinitely. With regard to the second embodiment of the present invention, FIG. 13 shows that the periodicity for the duty cycle in this simulation is about eight days. It should be noted that the swing of the high-yield species concentration 240 of Cyclotella is exaggerated for purpose of illustration. The densities 241 of Asterionella, Fragilaria, Synedra, and Tabellaria remain at minimal levels throughout the simulation such that they are not visible in FIG. 13. The high-yield species Cyclotella retains dominance and the lipid yield 243 remains consistently high around 65 g/m² per day. Therefore the overall lipid yield for the methods of FIG. 5 and FIG. 6 is greater than that for the prior art method of FIG. 10, whether in high-growth mode or dominant mode.

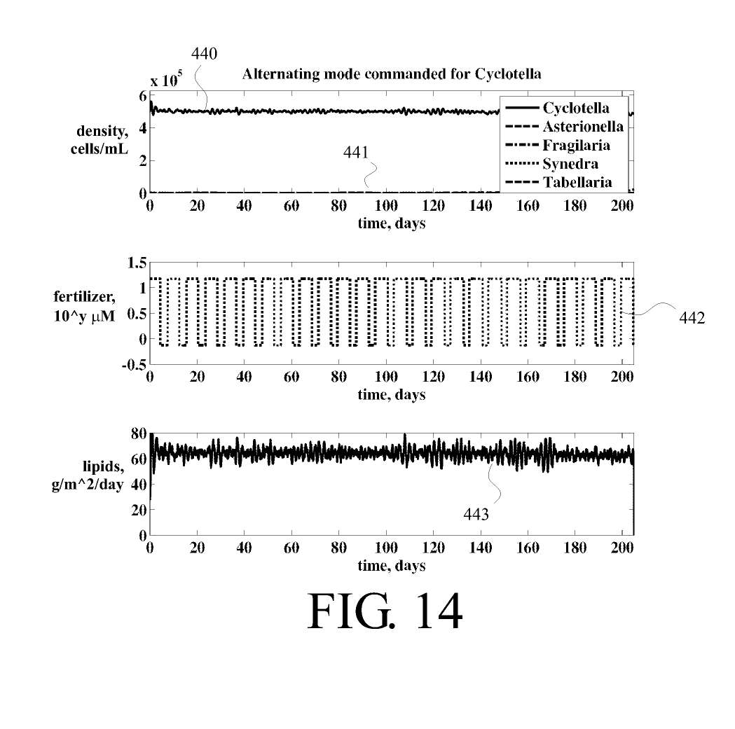

FIG. 14 shows simulation results using control methods of the third embodiment of the present invention applied to the same five fresh water algae species. In FIG. 14, the silicate concentration 442 alternates between high and low concentrations, which correspond to the high-growth and high-dominance modes respectively for the high-yield species Cyclotella. The proportional-integral control acts through the modal duty cycle (high-growth vs. high-dominance) to regulate the total species density. The periodicity for the duty cycle in this simulation is about eight days. The densities of Asterionella, Fragilaria, Synedra, and Tabellaria remain at minimal levels throughout the simulation such that they are not visible in FIG. 14. In FIG. 14, the high-yield species (Cyclotella) density 440 retains dominance over the invasive native species density 441 with the lipid yield 443 remaining consistently high around 65 g/m² per day. Therefore the overall lipid yield for the method of FIG. 7 is greater than that for the prior art method of FIG. 10, whether in high-growth mode or dominant mode.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method of controlling growth in an open algae cultivation system comprising a high-yield species and at least one invasive native species, the method comprising the steps of:
a) adjusting at least one parameter of the system to a first value such that a high-growth condition for the high-yield species is produced in the open algae cultivation system; and
b) adjusting the parameter to a second value different from the first value such that a high-dominance condition for the high-yield species is produced in the open algae cultivation system.

2. The method of claim 1, wherein the parameter is adjusted by controlling an element selected from the group consisting of inflow of brackish water, inflow of fresh water, inflow of CO₂ aeration, paddle speed, mix rate, agro-human waste inflow, harvest rate outflow, chemical additive inflow, and UV radiation.

3. The method of claim 1, wherein the parameter is adjusted by controlling fertilizer inflow.

4. The method of claim 1 further comprising the step of alternating between the high-growth condition and the high-dominance condition based on a feedback of a concentration of the high-yield species in the system.

5. The method of claim 4 further comprising the steps of:
c) adjusting from the first value to the second value when the concentration of the high-yield species reaches a lower limit; and
d) adjusting from the second value to the first value when the concentration of the high-yield species reaches an upper limit.

6. The method of claim 5 further comprising the step of maintaining the concentration of the high-yield species between the lower limit and the upper limit.

7. The method of claim 4, wherein the step of alternating further comprises the sub-step of using a periodic square wave, wherein a duty cycle is adjusted to maintain the high-yield species at a predetermined density by alternating between the high-growth condition and the high-dominance condition based on the feedback of the concentration of the high-yield species in the cultivation system.

8. The method of claim 7, wherein the step of alternating further comprises the sub-step of using proportional integral (PI) control to adjust the duty cycle based on the concentration of the high-yield species in the cultivation system.

9. The method of claim 1 further comprising the step of periodically harvesting a portion of the high-yield species from the cultivation system.

10. The method of claim 1 further comprising the step of continuously harvesting a portion of the high-yield species from the cultivation system.

11. A method of controlling growth in an open algae cultivation system comprising a high-yield species and at least one invasive native species, the method comprising the steps of:
a) maintaining the system at a high-growth condition for the high-yield species for a first portion of time; and
b) maintaining the system at a high-dominance condition for the high-yield species for a second portion of time.

12. The method of claim 11 further comprising the step of adjusting at least one parameter of the system to switch between the high-growth condition and the high-dominance condition.

13. The method of claim 12, wherein the parameter is adjusted by controlling an element selected from the group consisting of inflow of brackish water, inflow of fresh water, inflow of CO₂ aeration, paddle speed, mix rate, agro-human waste inflow, harvest rate outflow, chemical additive inflow, and UV radiation.

14. The method of claim 12, wherein the parameter is adjusted by controlling fertilizer inflow.

15. The method of claim 12 further comprising the steps of:
c) adjusting from a first value of the parameter to a second value of the parameter such that a high-dominance condition for the high-yield species is produced in the open algae cultivation system when the concentration of the high-yield species reaches a lower limit; and
d) adjusting from the second value to the first value such that a high-growth condition for the high-yield species is produced in the open algae cultivation system when the concentration of the high-yield species reaches an upper limit.

16. The method of claim 11 further comprising the step of alternating between the high-growth condition and the high-dominance condition based on a feedback of a concentration of the high-yield species in the system.

17. The method of claim 16, wherein the step of alternating further comprises the sub-step of using a periodic square wave, wherein a duty cycle is adjusted to maintain the high-yield species at a predetermined density by alternating between the high-growth condition and the high-dominance condition based on the feedback of the concentration of the high-yield species in the cultivation system.

18. The method of claim 17, wherein the step of alternating further comprises the sub-step of using proportional integral (PI) control to adjust the duty cycle based on the concentration of the high-yield species in the cultivation system.

19. The method of claim 11 further comprising the step of periodically harvesting a portion of the high-yield species from the cultivation system.

20. The method of claim 11 further comprising the step of continuously harvesting a portion of the high-yield species from the cultivation system.
